SEQUENCE LISTING (10 sequences)

SEQ ID NO: 1 - DNA, 69 bp, Artificial Sequence
  Synthetic primer
ccaacagagcagcagctacgggcagcagaacccttcttatgactcagtcagaagaggagcttggggcaa

SEQ ID NO: 2 - DNA, 70 bp, Artificial Sequence
  Synthetic primer
ccaacagagcagcagctacgggcagcagagttcactgctggcctataatacaacctcccacaccgaccaa

SEQ ID NO: 3 - DNA, 58 bp, Artificial Sequence
  Synthetic primer
catggatgaaggaccagatcttgatctagacccttcttatgactcagtcagaagagga

SEQ ID NO: 4 - DNA, 70 bp, Artificial Sequence
  Synthetic primer
catggatgaaggaccagatcttgatctaggttcactgctggcctataatacaacctcccacaccgaccaa

SEQ ID NO: 5 - DNA, 69 bp, Artificial Sequence
  Synthetic primer
ccaacagagcagcagctacgggcagcagaatttaccatatgagccccccaggagatcagcctggaccgg

SEQ ID NO: 6 - DNA, 19 bp, Artificial Sequence
  Synthetic primer
ctacgggcagcagaaccct

SEQ ID NO: 7 - DNA, 19 bp, Artificial Sequence
  Synthetic primer
cgggcagcagagttcactg

SEQ ID NO: 8 - DNA, 19 bp, Artificial Sequence
  Synthetic primer
tcttgatctagacccttct

SEQ ID NO: 9 - DNA, 19 bp, Artificial Sequence
  Synthetic primer
agatcttgatctaggttca

SEQ ID NO: 10 - DNA, 19 bp, Artificial Sequence
  Synthetic primer
ctacgggcagcagaattta

(19) United States
(12) Patent Application Publication
(10) Pub. No.: US 2013/0259927 A1
(43) Pub. Date: Oct. 3, 2013

(54) Ewing's Sarcoma Bifunctional shRNA Design

(71) Applicant: GRADALIS, INC., Carrollton, TX (US)

(72) Inventors: John J. Nemunaitis, Cedar Hill, TX (US); Donald Rao, Dallas, TX (US); Neil Senzer, Dallas, TX (US)

(73) Assignee: Gradalis, Inc., Carrollton, TX (US)

(21) Appl. No.: 13/855,109

(22) Filed: Apr. 2, 2013

Related U.S. Application Data
(60) Provisional application No. 61/619,077, filed on Apr. 2, 2012.

(51) Int. Cl. C12N 15/113

(52) U.S. Cl. CPC C12N 15/1135
USPC 424/450; 435/320.1; 514/44 R; 435/375

(57) ABSTRACT

The present invention includes compositions and methods of making and using an imaging label comprising an expression vector comprising a promoter; and a nucleic acid insert operably linked to the promoter, wherein the insert encodes one or more short hairpin RNAs (shRNA) capable of inhibiting an expression of a target gene sequence that is a EWS-FLI1 fusion gene, a EWSR1-ERG fusion gene, or both in Ewing's sarcoma via RNA interference; wherein the one or more shRNA comprise a bifunctional RNA molecule that activates a cleavage-dependent and a cleavage-independent RNA-induced silencing complex for reducing the expression level of the target gene.

### CROSS-REFERENCE TO RELATED APPLICATIONS

This application claims priority to U.S. Provisional Application Ser. No. 61/619,077 filed on Apr. 2, 2012, the entire contents of which are incorporated herein by reference.

### TECHNICAL FIELD OF THE INVENTION

The present invention relates in general to the field of gene-targeted cancer therapy, and more particularly, to the development of a bifunctional shRNA for a therapeutic RNA interference technology targeted towards Ewing's sarcoma family tumors (ESFTs).

### STATEMENT OF FEDERALLY FUNDED RESEARCH

None.

### REFERENCE TO A SEQUENCE LISTING

The present application includes a Sequence Listing which has been submitted in ASCII format via EFS-Web and is hereby incorporated by reference in its entirety. Said ASCII copy, created on Apr. 1, 2013, is named GRAD:1033 Sequence Listing.txt and is 3 KB in size.

### BACKGROUND OF THE INVENTION

Without limiting the scope of the invention, its background is described in connection with the gene therapies directed against Ewing's sarcoma.

U.S. Patent Application No. 20100167994 (Toretsky et al. 2010) discloses peptides and compounds that function as EWS-FLI1 protein inhibitors. The peptides and compounds have utility in the treatment of Ewing's sarcoma family of tumors. Also provided are methods of preparing the compounds and assays for identifying inhibitors of EWS-FLI1 protein.

U.S. Patent Application No. 20080280844 (Lessnick 2008) relates to methods and compositions for the detection and treatment of Ewing's sarcoma. In particular, the methods of detection relate to measuring in Ewing's sarcoma cells the expression of the NKX2.2 gene, as well as targets genes downstream of NKX2.2. The compositions and method of treatment for Ewing's sarcoma involve therapeutic agents that target the expression of the NKX2.2 gene or block the activity of the NKX2.2 protein. Also provided are methods of screening therapeutic agents that affect the expression of the NKX2.2 gene.

### SUMMARY OF THE INVENTION

The present invention includes a bifunctional shRNA design directed against Ewing's sarcoma and compositions and methods using the same for the treatment of Ewing's sarcoma family tumors (ESFTs). Certain embodiments include expression vectors comprising a promoter; and a nucleic acid insert operably linked to the promoter, wherein the insert encodes one or more short hairpin RNAs (shRNA) capable of inhibiting an expression of a target gene sequence that is a EWS-FLI1 fusion gene, a EWSR1-ERG fusion gene, or both in Ewing's sarcoma via RNA interference; wherein the one or more shRNA comprise a bifunctional RNA molecule that activates a cleavage-dependent and a cleavage-independent RNA-induced silencing complex for reducing the expression level of the target gene. In certain aspects, the target gene sequence may be a junction sequence of the EWS-FLI1 fusion gene or the EWSR1-ERG fusion gene; and/or may be at least one of SEQ ID NO: 1-10. In certain aspects, a sequence arrangement for the shRNA comprises a 5′ stem arm-19 nucleotide target (EWS-FLI1/EWSR1-ERG fusion gene or both)-TA-15 nucleotide loop-19 nucleotide target complementary sequence-3′ stem arm-Spacer-5′ stem arm-19 nucleotide target variant-TA-15 nucleotide loop-19 nucleotide target complementary sequence-3′ stem arm. Certain embodiments include a therapeutic delivery system comprising: a therapeutic agent carrier; and an expression vector comprising a promoter and a nucleic acid insert operably linked to the promoter encodes one or more short hairpin RNA (shRNA) capable inhibiting an expression of a target gene sequence that is a EWS-FLI1 fusion gene, a EWSR1-ERG fusion gene, or both in Ewing's sarcoma via RNA interference; wherein the one or more shRNA comprise a bifunctional RNA molecule that activates a cleavage-dependent and a cleavage-independent RNA-induced silencing complex for reducing the expression level of the target gene. The therapeutic agent carrier may, in certain aspects, be a compacted DNA nanoparticle, and the DNA nanoparticle may be compacted with one or more polycations, e.g., a 10 kDA polyethylene glycol (PEG)-substituted cysteine-lysine 3-mer peptide (CK₃₀PEG10k). The compacted DNA nanoparticles may be further encapsulated in a liposome; and the liposome may be a bilamellar invaginated vesicle (BIV); in certain aspects; the liposome is a reversibly masked liposome; the liposome may be decorated with one or more “smart” receptor targeting moieties, e.g., small molecule bivalent beta-turn mimics; and the therapeutic agent carrier may be a liposome. In certain aspects, the liposome is a bilamellar invaginated vesicle (BIV) decorated with one or more “smart” receptor targeting moieties, wherein the liposome is a reversibly masked liposome; the “smart” receptor targeting moieties are small molecule bivalent beta-turn mimics; and/or the target gene sequence is EWS-FLI1, EWSR1-ERG, SEQ ID NO: 1-10, or combinations or modifications thereof. Embodiments include methods to deliver one or more shRNAs to a target tissue expressing an EWS-FLI1 fusion gene, an EWSR1-ERG fusion gene, or both comprising the steps of preparing an expression vector comprising a promoter and a nucleic acid insert operably linked to the promoter that encodes the one or more shRNA, wherein the one or more shRNA are capable of inhibiting an expression of a target gene sequence that is a EWS-FLI1 fusion gene, a EWSR1-ERG fusion gene, or both in Ewing's sarcoma via RNA interference; combining the expression vector with a therapeutic agent carrier, wherein the therapeutic agent carrier is a liposome decorated with one or more “smart” receptor targeting moieties; and administering a therapeutically effective amount of the expression vector and therapeutic agent carrier complex to a patient in need thereof. In certain aspects, the therapeutic agent carrier may be a compacted DNA nanoparticle; the DNA nanoparticle may be compacted with one or more polycations, wherein the one or more polycations comprise a 10 kDA polyethylene glycol (PEG)-substituted cysteine-lysine 3-mer peptide (CK₃₀PEG10k) or a 30-mer lysine condensing peptide; the compacted DNA nanoparticles may be further encapsulated in a liposome, wherein the liposome is a bilamellar invaginated vesicle (BIV) and is decorated with one or more “smart” receptor targeting moieties; the one or more “smart” receptor targeting moieties may be small molecule bivalent beta-turn mimics; the liposome may be a reversibly masked liposome; the liposome may be a bilamellar invaginated vesicle (BIV); the one or more “smart” receptor targeting moieties may be small molecule bivalent beta-turn mimics; and/or the EWS-FLI1, EWSR1-ERG fusion gene or both may be selected from the group consisting of SEQ ID NO: 1-10. Certain embodiments include methods to inhibit an expression of a EWS-FLI1 fusion gene, an EWSR1-ERG fusion gene, or both in one or more target cells comprising the steps of: selecting the one or more target cells; and transfecting the target cell with a vector that expresses one or more short hairpin RNA (shRNAs) capable of inhibiting an expression of a EWS-FLI1 fusion gene, a EWSR1-ERG fusion gene, or both in the one or more target cells via RNA interference. In certain aspects, the shRNA incorporates siRNA (cleavage-dependent) and miRNA (cleavage-independent) motifs; the shRNA is both a cleavage-dependent and a cleavage-independent inhibitor of EWS-FLI1 fusion gene or EWSR1-ERG fusion gene expression; and/or the shRNA is further defined as a bifunctional shRNA. A sequence arrangement for the shRNA may comprise a 5′ stem arm-19 nucleotide target (EWS-FLI1/EWSR1-ERG fusion gene or both)-TA-15 nucleotide loop-19 nucleotide target complementary sequence-3′ stem arm-Spacer-5′ stem arm-19 nucleotide target variant-TA-15 nucleotide loop-19 nucleotide target complementary sequence-3′ stem arm. In various aspects, the EWS-FLI1, EWSR1-ERG fusion gene or both are selected from the group consisting of SEQ ID NO: 1, SEQ ID NO: 2, SEQ ID NO: 3, SEQ ID NO: 4, SEQ ID NO: 5, and combinations or modifications thereof. Embodiments include methods of suppressing a tumor cell growth, treating Ewing's sarcoma, or both in a human subject comprising the steps of identifying the human subject in need for suppression of the tumor cell growth, treatment of the Ewing's sarcoma or both; and administering a an expression vector in a therapeutic agent carrier complex to the human subject in an amount sufficient to suppress the tumor cell growth, treat the Ewing's sarcoma or both, wherein the expression vector expresses one or more shRNA capable inhibiting an expression of a target gene that is a EWS-FLI1 fusion gene, a EWSR1-ERG fusion gene, or both in the one or more target cells via RNA interference, wherein the one or more shRNA comprise a bifunctional RNA molecule that activates a cleavage-dependent and a cleavage-independent RNA-induced silencing complex for reducing the expression level of the target gene, wherein the inhibition results in an apoptosis, an arrested proliferation, or a reduced invasiveness of the tumor cells. In various aspects, a sequence arrangement for the shRNA may comprise a 5′ stem arm-19 nucleotide target (EWS-FLI1/EWSR1-ERG fusion gene or both)-TA-15 nucleotide loop-19 nucleotide target complementary sequence-3′ stem arm-Spacer-5′ stem arm-19 nucleotide target variant-TA-15 nucleotide loop-19 nucleotide target complementary sequence-3′ stem arm; and/or the EWS-FLI1, EWSR1-ERG fusion gene or both are selected from the group consisting of a sequence selected from SEQ ID NO: 1-10. In certain aspects, the therapeutic agent carrier is a compacted DNA nanoparticle or a reversibly masked liposome decorated with one or more “smart” receptor targeting moieties, the DNA nanoparticle is compacted with one or more polycations, wherein the one or more polycations is a 10 kDA polyethylene glycol (PEG)-substituted cysteine-lysine 3-mer peptide (CK₃₀PEG10k) or a 30-mer lysine condensing peptide; and/or the reversibly masked liposome is a bilamellar invaginated vesicle (BIV). In further aspects, the one or more “smart” receptor targeting moieties may be small molecule bivalent beta-turn mimics, and/or the compacted DNA nanoparticles are further encapsulated in a liposome.

### BRIEF DESCRIPTION OF THE DRAWINGS

For a more complete understanding of the features and advantages of the present invention, reference is now made to the detailed description of the invention along with the accompanying figures and in which:

FIGS. 1A and 1B are schematic representations showing the design of the bi-functional shRNAs of the present invention. FIG. 1A shows the sequence arrangement for a single target, and FIG. 1B shows the sequence arrangement for multiple targets.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

As used herein the term “nucleic acid” or “nucleic acid molecule” refers to polynucleotides, such as deoxyribonucleic acid (DNA) or ribonucleic acid (RNA), oligonucleotides, fragments generated by the polymerase chain reaction (PCR), and fragments generated by any of ligation, scission, endonuclease action, and exonuclease action. Nucleic acid molecules can be composed of monomers that are naturally-occurring nucleotides (such as DNA and RNA), or analogs of naturally-occurring nucleotides (e.g., α-enantiomeric forms of naturally-occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, phosphoramidate, and the like. The term “nucleic acid molecule” also includes so-called “peptide nucleic acids,” which comprise naturally-occurring or modified nucleic acid bases attached to a polyamide backbone. Nucleic acids can be either single stranded or double stranded.

The term “expression vector” as used herein in the specification and the claims includes nucleic acid molecules encoding a gene that is expressed in a host cell. Typically, an expression vector comprises a transcription promoter, a gene, and a transcription terminator. Gene expression is usually placed under the control of a promoter, and such a gene is said to be “operably linked to” the promoter. Similarly, a regulatory element and a core promoter are operably linked if the regulatory element modulates the activity of the core promoter. The term “promoter” refers to any DNA sequence which, when associated with a structural gene in a host yeast cell, increases, for that structural gene, one or more of 1) transcription, 2) translation or 3) mRNA stability, compared to transcription, translation or mRNA stability (longer half-life of mRNA) in the absence of the promoter sequence, under appropriate growth conditions.

The term “oncogene” as used herein refers to genes that permit the formation and survival of malignant neoplastic cells (Bradshaw, T. K.: Mutagenesis 1, 91-97 (1986).

As used herein the term “receptor” denotes a cell-associated protein that binds to a bioactive molecule termed a “ligand.” This interaction mediates the effect of the ligand on the cell. Receptors can be membrane bound, cytosolic or nuclear; monomeric (e.g., thyroid stimulating hormone receptor, beta-adrenergic receptor) or multimeric (e.g., PDGF receptor, growth hormone receptor, IL-3 receptor, GM-CSF receptor, G-CSF receptor, erythropoietin receptor and IL-6 receptor). Membrane-bound receptors are characterized by a multi-domain structure comprising an extracellular ligand-binding domain and an intracellular effector domain that is typically involved in signal transduction. In certain membrane-bound receptors, the extracellular ligand-binding domain and the intracellular effector domain are located in separate polypeptides that comprise the complete functional receptor.

The term “hybridizing” refers to any process by which a strand of nucleic acid binds with a complementary strand through base pairing.

The term “transfection” refers to the introduction of foreign DNA into eukaryotic cells. Transfection may be accomplished by a variety of means known to the art including, e.g., calcium phosphate-DNA co-precipitation, DEAE-dextran-mediated transfection, polybrene-mediated transfection, electroporation, microinjection, liposome fusion, lipofection, protoplast fusion, retroviral infection, and biolistics.

As used herein the term “bi-functional” refers to a shRNA having two mechanistic pathways of action, that of the siRNA and that of the miRNA. The term “traditional” shRNA refers to a DNA transcription derived RNA acting by the siRNA mechanism of action. The term “doublet” shRNA refers to two shRNAs, each acting against the expression of two different genes but in the “traditional” siRNA mode.

As used herein, the term “liposome” refers to a closed structure composed of lipid bilayers surrounding an internal aqueous space. The term “polycation” as used herein denotes a material having multiple cationic moieties, such as quaternary ammonium radicals, in the same molecule and includes the free bases as well as the pharmaceutically-acceptable salts thereof.

The present invention includes a bi-shRNA developed for the purpose of concurrently inducing translational repression and post-transcriptional mRNA degradation of its target. The bi-shRNA of the present invention is directed against Ewing's sarcoma family tumors (ESFTs)

The major forms of ESFTs are EWS-FLI1 fusion genes which constitutes more than 85% of ESFTs. EWS-FLI1 is a chimeric ETS transcription factor due to chromosomal rearrangement. Chromosomal translocation results in the formation of gene fusion between the EWSK1 locus and an ETS transcription factor gene FLI1 t(11;22)(q24;q12). The rearranged fusion genes can occur between different exons resulting in at least four types (1-4) of mRNAs. In addition, 10-15% of EFSTs involve translocation between the EWSR1 locus and an ETS transcription factor gene ERG t(21;22)(q22;q12) generates EWSR1-ERG fusion.

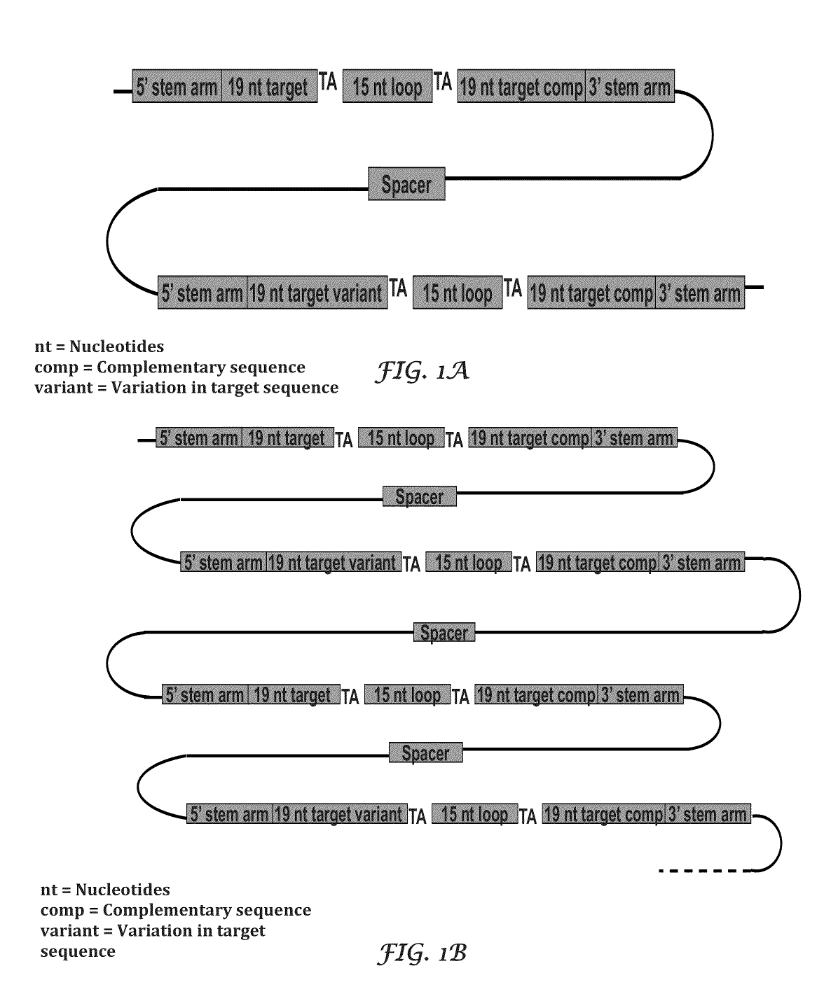

bi-shRNA: The present inventors have pioneered a unique RNAi platform known as bi-functional shRNA. Conceptually, RNAi can be achieved through shRNA-loaded RISCs to promote cleavage-dependent or cleavage-independent mRNA knockdown. Concomitant expression of both configurations of shRNAs (hence the nomenclature, bi-functional shRNA) to promote loading onto multiple types of RISCs has been shown by the present inventors to achieve more effective target gene knockdown at a more rapid onset of silencing (rate of mRNA and protein turnover notwithstanding) with greater durability as compared with siRNA. The basic design of the bi-functional shRNA expression unit comprises two stem-loop shRNA structures; one composed of fully matched passenger and guide strands for cleavage-dependent RISC loading, and a second stem-loop with a mismatched passenger strand (at positions 9-12) for cleavage-independent RISC loading. This bi-functional design is, much more efficient for two reasons; first, the bi-functional promotes guide strand loading onto distinct RISC types, hence promoting mRNA targeting; second, the presence of cleavage-dependent and cleavage-independent RISCs against the same target mRNA promotes silencing by both degradation and translational inhibition/sequestration processes. The potent gene knockdown effector achieves spatial and temporal control by the multiplexed shRNAs under the control of a single pol II promoter. The platform designed by the present inventors mimics the natural process. Multiple studies by others and the literature support the approach of the present inventors. A schematic representation of the bi-functional shRNA design against a single or against multiple targets is shown in FIGS. 1A and 1B, respectively.

Using a miR30-scaffold, the inventors have produced novel bifunctional (bi-shRNA) against the microtubule remodeling oncoprotein stathmin (STMN1, oncoprotein 18, prosolin, p19, op 18). Bi-shRNA^{STMN1} demonstrated more effective silencing activity as compared with siRNAs to the same target site. STMN1 is critically involved in mitotic spindle formation [71, 72]. The bi-shRNA^{STMN1} construct described previously by the present inventors demonstrated safe, effective target knockdown and significant dose advantage in tumor cell killing when compared to siRNA to the same target. The inventors have validated intracellular transcription and processing of both mature and effector molecules (dsRNA with complete matching strands and dsRNA with specified mismatches), using a RT-PCR method that can discriminate between matched and mismatched passenger strands [73]. Most cancer cells have high Drosha and Dicer expression. There has been controversy regarding endogenous Dicer levels in cancer cells [74]. Nonetheless, most studies have indicated that sh or bi-sh RNAi knockdown is highly effective in cancer cells with even low levels of Dicer expression [75]. The present inventors confirmed the expression of the predicted matched and mismatched shRNAs that correspond to mature miRNA/siRNA components to bi-shRNA^{STMN1}, as opposed to only having the fully matched passenger stranded in control siRNA^{STMN1} treated cells [76]. To further support the mechanism of the bi-sh RNA^{STMN1}, the findings of the studies in the present invention with the 5′ RACE method have confirmed the presence of STMN-1 cleavage products with expected sequence corresponding to the target cleavage site of the siRNA (matched) component of the bi-shRNA^{STMN1}. Effective knockdown (93%) of STMN1 expressive tumor cells was observed, reflecting the outcome of both cleavage-dependent and independent-mediated knockdowns of the bi-shRNA^{STMN1}. Furthermore, STMN1 mRNA kinetics observed following knockdown with the separate component cleavage-dependent (GBI-1) and cleavage-independent (GBI-3) vectors compared to bi-shRNA^{STMN1} (GBI-2) were consistent with predicted mechanism.

Liposomal delivery system: The liposomal delivery system previously validated by the inventors involved 1,2-dioleoyl-3-trimethyl-ammoniopropane (DOTAP) and cholesterol [77]. This formulation combines with DNA to form complexes that encapsulate nucleic acids within bilamellar invaginated vesicles (liposomal BIVs). One of the inventors has optimized several features of the BIV delivery system for improved delivery of RNA, DNA, and RNAi plasmids. The liposomal BIVs are fusogenic, thereby bypassing endocytosis mediated DNA cell entry, which can lead to nucleic acid degradation [78] and TLR mediated off-target effects. This liposomal delivery system has been used successfully in clinical trial by the present inventors and others [79-83]. Cumulative studies over the last decade indicate that the optimized delivery vehicle needs to be a stealthed (commonly achieved by PEGylation) nanoparticle with a zeta potential of ≦10 mV for efficient intravascular transport [84-86] in order to minimize nonspecific binding to negatively-charged serum proteins such as serum albumin (opsonization) [87]. Incorporation of targeting moieties such as antibodies and their single chain derivatives (scFv), carbohydrates, or peptides may further enhance transgene localization to the target cell.

The present inventors have created targeted delivery of the complexes in vivo without the use of PEG thereby avoiding an excessively prolonged circulatory half-life [86, 88-90]. While PEGylation is relevant for DNA or siRNA oligonucleotide delivery to improve membrane permeability, this approach has been shown by the inventors and others to cause steric hindrance in the BIV liposomal structures, resulting in inefficient DNA encapsulation and reduced gene expression. Furthermore, PEGylated complexes enter the cell predominantly through the endocytic pathway, resulting in degradation of the bulk of the nucleic acid in the lysosomes. While PEG provides extremely long half-life in circulation, this has created problems for patients as exemplified by doxil, a PEGylated liposomal formulation that encapsulates the cytotoxic agent doxorubicin [90-92]. Attempts to add ligands to doxil for delivery to specific cell surface receptors (e.g. HER2/neu) have not enhanced tumor-specific delivery [93].

Based on this reasoning, the BIVs of the present invention were produced with DOTAP, and synthetic cholesterol using proprietary manual extrusion process [94]. Furthermore, the delivery was optimized using reversible masking technology. Reversible masking utilizes small molecular weight lipids (about 500 Mol. Wt. and lower; e.g. n-dodecyl-β-D-maltopyranoside) that are uncharged and, thereby, loosely associated with the surface of BIV complexes, thereby temporarily shielding positively charged BIV complexes to bypass non-targeted organs. These small lipids are removed by shear force in the bloodstream. By the time they reach the target cell, charge is re-exposed (optimally ˜45 mV) to facilitate entry.

One reason that the BIV delivery system is uniquely efficient is because the complexes deliver therapeutics into cells by fusion with the cell membrane and avoid the endocytic pathway. The two major entry mechanisms of liposomal entry are via endocytosis or direct fusion with the cell membrane. The inventors found that nucleic acids encapsulated in BIV complexes delivered both in vitro and in vivo enter the cell by direct fusion and that the BIVs largely avoid endosomal uptake, as demonstrated in a comparative study with polyethylene-amine (PEI) in mouse alveolar macrophages. PEI is known to be rapidly and avidly taken up into endosomes, as demonstrated by the localization of ≧95% of rhodamine labeled oligonucleotides within 2-3 hrs post-transfection [95-97].

Cancer targeted delivery with decorated BIVs: Recently, Bartlett and Davis showed that siRNAs that were delivered systemically by tumor-targeted nanoparticles (NPs) were significantly more effective in inhibiting the growth of subcutaneous tumors, as compared to undecorated NPs [98]. Targeted delivery did not significantly impact pharmacokinetics or biodistribution, which remain largely an outcome of the EPR (enhanced permeability and retention) effect [95], but appeared to improved transgene expression through enhanced cellular uptake [95-97].

Indeed, a key “missing piece” in development of BIVs for therapeutic use has been the identification of such non-immunogenic ligands that can be placed on the surface of BIV-complexes to direct them to target cells. While it might be possible to do this with small peptides that are multimerized on the surface of liposomes, these can generate immune responses after repeated injections. Other larger ligands including antibodies, antibody fragments, proteins, partial proteins, etc. are far more refractory than using small peptides for targeted delivery on the surface of liposomes. The complexes of the present invention are thus unique insofar as they not only penetrate tight barriers including tumor vasculature endothelial pores and the interstitial pressure gradient of solid tumors [99], but also target tumor cells directly. Therefore, the therapeutic approach of the present invention is not limited to delivery solely dependent on the EPR effect but targets the tumor directly [100-102].

Small molecules designed to bind proteins selectively can be used with the present invention. Importantly, the small molecules prepared are “bivalent” so they are particularly appropriate for binding cell surface receptors, and resemble secondary structure motifs found at hot-spots in protein-ligand interactions. The Burgess group has had success in designing bivalent beta-turn mimics that have an affinity for cell surface receptors [103-105]. The strategy has been adapted by the present inventors to give bivalent molecules that have hydrocarbon tails, and we prepared functionalized BIV complexes from these adapted small molecules. An efficient high throughput technology to screen the library was developed and run.

Compacted DNA Nanoparticles: Safe and Efficient DNA Delivery in Post-Mitotic Cells: The Copernicus nucleic acid delivery technology is a non-viral synthetic and modular platform in which single molecules of DNA or siRNA are compacted with polycations to yield nanoparticles having the minimum possible volume [106]. The polycations optimized for in vivo delivery is a 10 kDa polyethylene glycol (PEG) modified with a peptide comprising a N-terminus cysteine and 30 lysine residues (CK₃₀PEG10k). The shape of these complexes is dependent in part on the lysine counterion at the time of DNA compaction [107]. The minimum cross-sectional diameter of the rod nanoparticles is 8-11 nm irrespective of the size of the payload plasmid, whereas for ellipsoids the minimum diameter is 20-22 nm for typical expression plasmids (FIG. 7A) [107]. Importantly, these DNA nanoparticles are able to robustly transfect non-dividing cells in culture. Liposome mixtures of compacted DNA generate over 1,000-fold enhanced levels of gene expression compared to liposome naked DNA mixtures (FIG. 7B). Following in vivo dosing, compacted DNA robustly transfects post-mitotic cells in the lung [108], brain [109, 110], and eye [111, 112]. In each of these systems the remarkable ability of compacted DNA to transfect post-mitotic cells appears to be due to the small size of these nanoparticles, which can cross the cross the 25 nm nuclear membrane pore [106].

One uptake mechanism for these DNA nanoparticles is based on binding to cell surface nucleolin (26 nm K_{D}), with subsequent cytoplasmic trafficking via a non-degradative pathway into the nucleus, where the nanoparticles unravel releasing biologically active DNA [113]. Long-term in vivo expression has been demonstrated for as long as 1 year post-gene transfer. These nanoparticles have a benign toxicity profile and do not stimulate toll-like receptors thereby avoiding toxic cytokine responses, even when the compacted DNA has hundreds of CpG islands and are mixed with liposomes, no toxic effect has been observed [114,115]. DNA nanoparticles have been dosed in humans in a cystic fibrosis trial with encouraging results, with no adverse events attributed to the nanoparticles and with most patients demonstrating biological activity of the CFTR protein [116].

EWS-FLI1 Type 1-4 constitutes approximately 85% of all ESFTs. Junction sequence of Type 1-4 are presented herein below (underlined=EWSR1, non-underlined=FLI1):

(SEQ ID NO: 1)
CCAACAGAGCAGCAGCTACGGGCAGCAGAACCCTTCTTATGACTCAGTC
AGAAGAGGAGCTTGGGGCAA
(SEQ ID NO: 2)
CCAACAGAGCAGCAGCTACGGGCAGCAGAGTTCACTGCTGGCCTATAAT
ACAACCTCCCACACCGACCAA
(SEQ ID NO: 3)
CATGGATGAAGGACCAGATCTTGATCTAGACCCTTCTTATGACTCAGTC
AGAAGAGGA
(SEQ ID NO: 4)
CATGGATGAAGGACCAGATCTTGATCTAGGTTCACTGCTGGCCTATAAT
ACAACCTCCCACACCGACCAA

EWS-ERG constitutes approximately 10% of all ESFTs. Junction sequence of EWS/ERG is described herein below (underlined=EWSR1, non-underlined=ERG):

(SEQ ID NO: 5)
CCAACAGAGCAGCAGCTACGGGCAGCAGAATTTACCATATGAGCCCCCC
AGGAGATCAGCCTGGACCGG

The bi-shRNA of the present invention is designed keeping some design considerations in mind: (i) 4 sets of bifunctionals (Table 1) were prepared and tested individually and were then put together, (ii) The junction point was placed at the middle of the seed region for specificity. This design (which is different from majority of published siRNAs) will have strong specificity for the fusion mRNA without affecting the normal non-rearranged EWSR1, FLT1 or ERG transcripts, (iii) For type II and type III, because of homologous sequence at EWS, more extended FLI1 sequence has to be included. One could further test by moving around the region, and (iv) Although the in silico screen for the sequence related off-target effect indicated low hits for each bi-sh design, but all five bi-shRNAs have different potential off-target hits; if we string them together, the potential off-targets will essentially add up. Type IV is the one with least number of potential off-targets.

TABLE 1
Target sequences for bi-shRNAs against ESFTS

| Type | Target sequences | ID |
|---|---|---|
| I | CTACGGGCAGCAGAACCCT | SEQ ID NO: 6 |
| II | CGGGCAGCAGAGTTCACTG | SEQ ID NO: 7 |
| III | TCTTGATCTAGACCCTTCT | SEQ ID NO: 8 |
| IV | AGATCTTGATCTAGGTTCA | SEQ ID NO: 9 |
| ERG | CTACGGGCAGCAGAATTTA | SEQ ID NO: 10 |

The construction of a novel bi-shRNA therapeutic of the present invention represents a state-of-the art approach that can reduce the effective systemic dose needed to achieve an effective therapeutic outcome through post-transcriptional gene knockdown. Effective and clinically applicable delivery approaches are in place that can be rapidly transitioned for systemic targeting of ESFTs.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

U.S. Patent Application No. 20100167994: Targeting of EWS-FLI1 as Anti-Tumor Therapy.
U.S. Patent Application No. 20080280844: Methods and Compositions for the Diagnosis and Treatment of Ewing's Sarcoma.

What is claimed is:

1. An expression vector comprising:
a promoter; and
a nucleic acid insert operably linked to the promoter,
wherein the insert encodes one or more short hairpin RNAs (shRNA) capable of inhibiting an expression of a target gene sequence that is a EWS-FLI1 fusion gene, a EWSR1-ERG fusion gene, or both in Ewing's sarcoma via RNA interference;
wherein the one or more shRNA comprise a bifunctional RNA molecule that activates a cleavage-dependent and a cleavage-independent RNA-induced silencing complex for reducing the expression level of the target gene.

2. The expression vector of claim 1, wherein the target gene sequence is a junction sequence of the EWS-FLI1 fusion gene or the EWSR1-ERG fusion gene.

3. The expression vector of claim 1, wherein the target gene sequence is at least one of SEQ ID NO: 1-10.

4. The expression vector of claim 1, wherein a sequence arrangement for the shRNA comprises a 5′ stem arm-19 nucleotide target (EWS-FLI1/EWSR1-ERG fusion gene or both)-TA-15 nucleotide loop-19 nucleotide target complementary sequence-3′ stem arm-Spacer-5′ stem arm-19 nucleotide target variant-TA-15 nucleotide loop-19 nucleotide target complementary sequence-3′stem arm.

5. A therapeutic delivery system comprising:
a therapeutic agent carrier; and
an expression vector comprising a promoter and a nucleic acid insert operably linked to the promoter encodes one or more short hairpin RNA (shRNA) capable inhibiting an expression of a target gene sequence that is a EWS-FLI1 fusion gene, a EWSR1-ERG fusion gene, or both in Ewing's sarcoma via RNA interference;
wherein the one or more shRNA comprise a bifunctional RNA molecule that activates a cleavage-dependent and a cleavage-independent RNA-induced silencing complex for reducing the expression level of the target gene.

6. The delivery system of claim 5, wherein the therapeutic agent carrier is a compacted DNA nanoparticle.

7. The delivery system of claim 6, wherein the DNA nanoparticle is compacted with one or more polycations.

8. The delivery system of claim 6, wherein the one or more polycations is a 10 kDA polyethylene glycol (PEG)-substituted cysteine-lysine 3-mer peptide (CK₃₀PEG10k).

9. The delivery system of claim 6, wherein the compacted DNA nanoparticles are further encapsulated in a liposome.

10. The delivery system of claim 9, wherein the liposome is a bilamellar invaginated vesicle (BIV).

11. The delivery system of claim 9, wherein the liposome is a reversibly masked liposome.

12. The delivery system of claim 9, wherein the liposome is decorated with one or more “smart” receptor targeting moieties.

13. The delivery system of claim 12, wherein the one or more “smart” receptor targeting moieties are small molecule bivalent beta-turn mimics.

14. The delivery system of claim 5, wherein the therapeutic agent carrier is a liposome.

15. The delivery system of claim 14, wherein the liposome is a bilamellar invaginated vesicle (BIV) decorated with one or more “smart” receptor targeting moieties, wherein the liposome is a reversibly masked liposome.

16. The delivery system of claim 15, wherein the “smart” receptor targeting moieties are small molecule bivalent beta-turn mimics.

17. The delivery system of claim 5, wherein the target gene sequence is EWS-FLI1, EWSR1-ERG, SEQ ID NO: 1-10, or combinations or modifications thereof.

18. A method to deliver one or more shRNAs to a target tissue expressing an EWS-FLI1 fusion gene, an EWSR1-ERG fusion gene, or both comprising the steps of:
preparing an expression vector comprising a promoter and a nucleic acid insert operably linked to the promoter that encodes the one or more shRNA, wherein the one or more shRNA are capable of inhibiting an expression of a target gene sequence that is a EWS-FLI1 fusion gene, a EWSR1-ERG fusion gene, or both in Ewing's sarcoma via RNA interference;
combining the expression vector with a therapeutic agent carrier, wherein the therapeutic agent carrier is a liposome decorated with one or more “smart” receptor targeting moieties; and
administering a therapeutically effective amount of the expression vector and therapeutic agent carrier complex to a patient in need thereof.

19. The method of claim 18, wherein the therapeutic agent carrier is a compacted DNA nanoparticle.

20. The method of claim 19, wherein the DNA nanoparticle is compacted with one or more polycations, wherein the one or more polycations comprise a 10 kDA polyethylene glycol (PEG)-substituted cysteine-lysine 3-mer peptide (CK₃₀PEG10k) or a 30-mer lysine condensing peptide.

21. The method of claim 19, wherein the compacted DNA nanoparticles are further encapsulated in a liposome, wherein the liposome is a bilamellar invaginated vesicle (BIV) and is decorated with one or more “smart” receptor targeting moieties.

22. The method of claim 21, wherein the one or more “smart” receptor targeting moieties are small molecule bivalent beta-turn mimics.

23. The method of claim 21, wherein the liposome is a reversibly masked liposome.

24. The method of claim 18, wherein the liposome is a bilamellar invaginated vesicle (BIV).

25. The method of claim 18, wherein the liposome is a reversibly masked liposome.

26. The method of claim 18, wherein the one or more “smart” receptor targeting moieties are small molecule bivalent beta-turn mimics.

27. The method of claim 18, wherein the EWS-FLI1, EWSR1-ERG fusion gene or both are selected from the group consisting of SEQ ID NO: 1-10.

28. A method to inhibit an expression of a EWS-FLI1 fusion gene, an EWSR1-ERG fusion gene, or both in one or more target cells comprising the steps of:
selecting the one or more target cells; and
transfecting the target cell with a vector that expresses one or more short hairpin RNA (shRNAs) capable of inhibiting an expression of a EWS-FLI1 fusion gene, a EWSR1-ERG fusion gene, or both in the one or more target cells via RNA interference.

29. The method of claim 28, wherein the shRNA incorporates siRNA (cleavage-dependent) and miRNA (cleavage-independent) motifs.

30. The method of claim 28, wherein the shRNA is both a cleavage-dependent and a cleavage-independent inhibitor of EWS-FLI1 fusion gene or EWSR1-ERG fusion gene expression.

31. The method of claim 28, wherein the shRNA is further defined as a bifunctional shRNA.

32. The method of claim 28, wherein a sequence arrangement for the shRNA comprises a 5′ stem arm-19 nucleotide target (EWS-FLI1/EWSR1-ERG fusion gene or both)-TA-15 nucleotide loop-19 nucleotide target complementary sequence-3′ stem arm-Spacer-5′ stem arm-19 nucleotide target variant-TA-15 nucleotide loop-19 nucleotide target complementary sequence-3′ stem arm.

33. The method of claim 28, wherein the EWS-FLI1, EWSR1-ERG fusion gene or both are selected from the group consisting of SEQ ID NO: 1, SEQ ID NO: 2, SEQ ID NO: 3, SEQ ID NO: 4, SEQ ID NO: 5, and combinations or modifications thereof.

34. A method of suppressing a tumor cell growth, treating Ewing's sarcoma, or both in a human subject comprising the steps of:
identifying the human subject in need for suppression of the tumor cell growth, treatment of the Ewing's sarcoma or both; and
administering a an expression vector in a therapeutic agent carrier complex to the human subject in an amount sufficient to suppress the tumor cell growth, treat the Ewing's sarcoma or both, wherein the expression vector expresses one or more shRNA capable inhibiting an expression of a target gene that is a EWS-FLI1 fusion gene, a EWSR1-ERG fusion gene, or both in the one or more target cells via RNA interference;
wherein the one or more shRNA comprise a bifunctional RNA molecule that activates a cleavage-dependent and a cleavage-independent RNA-induced silencing complex for reducing the expression level of the target gene;
wherein the inhibition results in an apoptosis, an arrested proliferation, or a reduced invasiveness of the tumor cells.

35. The method of claim 34, wherein a sequence arrangement for the shRNA comprises a 5′ stem arm-19 nucleotide target (EWS-FLI1/EWSR1-ERG fusion gene or both)-TA-15 nucleotide loop-19 nucleotide target complementary sequence-3′ stem arm-Spacer-5′ stem arm-19 nucleotide target variant-TA-15 nucleotide loop-19 nucleotide target complementary sequence-3′stem arm.

36. The method of claim 34, wherein the EWS-FLI1, EWSR1-ERG fusion gene or both are selected from the group consisting of a sequence selected from SEQ ID NO: 1-10.

37. The method of claim 34, wherein the therapeutic agent carrier is a compacted DNA nanoparticle or a reversibly masked liposome decorated with one or more “smart” receptor targeting moieties.

38. The method of claim 37, wherein the DNA nanoparticle is compacted with one or more polycations, wherein the one or more polycations is a 10 kDA polyethylene glycol (PEG)-substituted cysteine-lysine 3-mer peptide (CK₃₀PEG10k) or a 30-mer lysine condensing peptide

39. The method of claim 37, wherein the reversibly masked liposome is a bilamellar invaginated vesicle (BIV).

40. The method of claim 37, wherein the one or more “smart” receptor targeting moieties are small molecule bivalent beta-turn mimics.

41. The method of claim 37, wherein the compacted DNA nanoparticles are further encapsulated in a liposome.
